(19) United States
(12) Patent Application Publication
(10) Pub. No.: US 2012/0192877 A1
(43) Pub. Date: Aug. 2, 2012

(54) HANDWICH MITT

(76) Inventor: BERNADETTE CHAVES, Kaneohe, HI (US)

(21) Appl. No.: 13/019,406

(22) Filed: Feb. 2, 2011

(51) Int. Cl. A61F 5/37

(52) U.S. Cl. 128/879

(57) ABSTRACT

A dual cushion restraint mitt comprising a first cushion positioned above a hand of a user; a second cushion attached to the first cushion at a seam, where the second cushion rests beneath the hand; a portion of netting fastened to the first cushion and attached at the seam, wherein the portion of netting wraps around the first cushion and the second cushion; and a cuff attached to an end of the portion of netting opposite to the seam, wherein the cuff wraps around a wrist of the user to keep the portion of netting in place while the first cushion and the second cushion sandwich the hand to restrain and to prevent the user from removing life sustaining equipment.

### BACKGROUND OF THE INVENTION

1. Field of the Invention

The present invention relates to a restraint glove to immobilize a user's hand, wherein the glove includes a pair of cushions that sandwich the hand to prevent gripping and damage during use.

2. Description of Related Art

Restraint mitts are a commonly used by health care professionals a to immobilize a person's hands. Most of the time a hospital patent is connected to various life sustaining tubes, for example vital lines, IVs and the like, to maintain and monitor their bodily functions. Many times the patient attempts to pull or dislodge these tubes especially if they are agitated, frustrated, disoriented or in pain. This is problematic because without the tubes they will not receive the proper treatment from the hospital. Likewise, law enforcement officers utilize restraint mitts to secure the hands of aggressive or violent detainees. The officers must protect themselves, the person and surrounding property from damage if the person becomes violent or destructive.

Currently, there are limited types of restraint mitts that are easy to maneuver and prevent slippage. Many of the available restraint mitts are difficult to place on the user's hand either due to small hand openings or multiple pieces, or they cause sweating and discomfort to the user while in place. Some improvements have been made to the restraint mitts but they do not cure all difficulties.

For example U.S. Pat. No. 3,476,108 by A. G. Matukas discloses a hand restraint wherein the hand restraint includes a mitt to accept a hand and is then secured around the wrist with strips of tape. The hand restraint enables the user to bend their hand along with the mitt while in use. This could be hazardous by still enabling the person to pull an IV out of their arm.

U.S. Pat. No. 4,887,616 by Etienette Baijnath discloses a restraint mitt for restraining wearer's hand and arm wherein a mitt is provided to enable the user to move their hands while inside the mitt and a strap to restrain the person's arms. The outer portion of the mitt is immobile when the user opens and closes their hands, because the outer portion or shell is made from a hard or rigid material. While the restraint mitt does prevent any sort of grabbing, the shell may be damaging to other persons and property if the person becomes angry and uses the mitt as a weapon.

It would be beneficial in the art to provide a hand restraint that immobilizes a person's hand while still being comfortable to the wearer. It would also be desirable to provide a hand restraint that allows for quick and easy placement of the restraint.

### SUMMARY OF THE INVENTION

An object of the present invention is to provide a hand restraint that immobilizes a wearer's hand to prevent them from removing life sustaining equipment or injuring others and themselves.

Another object of the present invention is to provide a hand restraint that includes two soft cushions to sandwich the hand for a comfortable feel.

Another object of the present invention is to provide a hand restraint that allows security and breathability because even though the hand has restricted movement air can still flow to the hand and prevent sweating or sores on the skin.

In view of the foregoing disadvantages inherent in the prior art, the purpose of the present disclosure is to provide a dual cushion restraint mitt comprising a first cushion positioned above a hand of a user; a second cushion attached to the first cushion at a seam, where the second cushion rests beneath the hand; a portion of netting fastened to the first cushion and attached at the seam, wherein the portion of netting wraps around the first cushion and the second cushion; and a cuff attached to an end of the portion of netting opposite to the seam, wherein the cuff wraps around a wrist of the user to keep the portion of netting in place while the first cushion and the second cushion sandwich the hand to restrain and to prevent the user from removing life sustaining equipment.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a perspective view of a handwich mitt in accordance with an exemplary embodiment of the present invention;

FIG. 2 depicts a perspective view of a handwich mitt in a closed position in accordance with an exemplary embodiment of the present invention; and

FIG. 3 depicts a side view of a handwich mitt in accordance with an exemplary embodiment of the present invention.

Like reference numerals refer to like parts throughout the description of several views of the drawings.

### DETAILED DESCRIPTION OF THE DRAWINGS

The present invention relates to a restraint glove to immobilize a user's hand, wherein the glove includes a pair of cushions that sandwich the hand to prevent gripping and damage during use. The present invention provides a handwich mitt or a dual cushion restraint mitt which prevents a user from grabbing or removing vital lines, IVs and tubes while in a hospital or under medical care. The handwich mitt utilizes two round cushions to sandwich the hand inside the mitt. Netting wraps around the cushions to secure them in place while preventing the hand from escaping the sides of the cushions. The netting is fastened to the user's wrist with an attached cuff to ensure that the cushions and the netting stay in place and do not slide during movement. When using the handwich mitt, the hands receive ample ventilation because the netting allows air to pass through the sides of the mitt for greater comfort and breathability. The handwich mitt may be available in various sizes to accommodate users from child to adult to comfortably immobilize the user's hands.

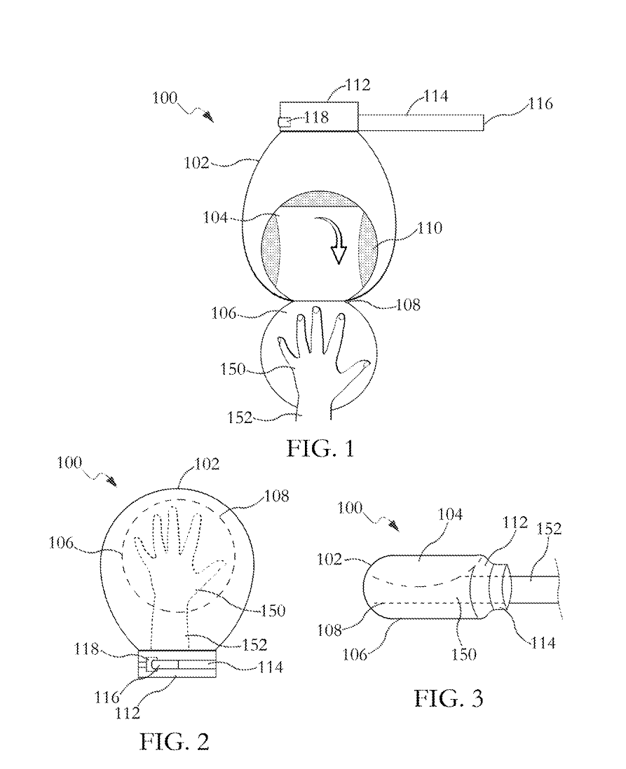

Tuning now descriptively to the drawings, referring to FIG. 1, a perspective view of a handwich mitt (100) is shown in accordance with an exemplary embodiment of the present invention. The handwich mitt (100) includes a first cushion (104) and a second cushion (106) attached together at a seam (108). The first cushion (104) and the second cushion (106) may be round or squared pillows that are compressible when pressed and then return to their original shape once the pressing stops. The second cushion (106) is positioned beneath a user's hand (150) or against the palm. During use, the first cushion (104) rests above the hand (150) and presses against the back of the hand (150) creating a sandwich effect. The first cushion (104) and the second cushion (106) may be made from a soft foam material, a cotton stuffed pillow, a synthetic padding or another similar material.

Fastened around the first cushion (104) and attached to the second cushion (106) at the seam (108) is a portion of netting (102). The portion of netting (102) wraps around the first cushion (104) and the second cushion (106) when the two cushions (104, 106) are placed around the hand (150). The portion of netting (150) secures the two cushions (104, 106) around the hand (150) to prevent the hand (150) from sliding or escaping the handwich mitt (100). By wrapping the cushions (104, 106) the portion of netting (102) ensures that the user cannot grab or remove any life supporting equipment. Additionally, the portion of netting (102) provides breathability against the hand (150) for greater comfort and to prevent sweating or sores on the skin.

Attached to the first cushion (104) may be a plurality of foam pieces (110). The plurality of foam pieces (110) are arranged with two foam pieces on the lateral portions of the first cushion (104) and at a portion opposite the seam (108). The plurality of foam pieces (110) provide additional cushioning to the handwich mitt (100) and additional barriers to prevent the hand (150) from escaping or losing its positioning between the cushions (104, 106).

At an end of the portion of netting (102) opposite where it attaches to the seam (108) is a cuff (112). The cuff (112) may be lined with a soft material like cotton, lamb's wool or synthetic stuffing for a soft and soothing feel. The cuff (112) wraps around the user's wrist (152) and a portion of the second cushion (106) when the first cushion (104) is positioned over the hand (150). To secure the cuff (112) around the wrist (152) the user may use a strap (114) and an attachment means. The strap (114) may be elastic to stretch and tightly fit around the wrist (152). The attachment means may a “D” ring (118) to engage the strap (114), a snap or a hook and loop fastener (more commonly known as Velcro®). To use the “D” ring (118) an end (116) of the strap (114) loops through the “D” ring (118) and is pulled to tighten the cuff (112) around the wrist (152). Beneath the end (116) may be the hook and loop fastener to cinch the cuff (112) around the wrist (152). The cuff (112) also secures the portion of netting (102) around the cushions (104, 106) so that the handwich mitt (100) remains stable around the hand (150).

Referring now to FIG. 2, a perspective view of the handwich mitt (100) is shown in a closed position in accordance with an exemplary embodiment of the present invention. The handwich mitt (100) may transition between an open position (as shown in FIG. 1) to a closed position (as shown in FIG. 2). In the open position the first cushion (104) and the second cushion (106) are positioned adjacent to each other merely touching at the seam (108). In the closed position the first cushion (104) is positioned above the second cushion (106) sandwiching the hand (150). When in the closed position the portion of netting (102) wraps around the cushions (104, 106) for a tight yet comfortable fit around the user's hand (150).

Referring to FIG. 3, a side view of the handwich mitt (100) is shown in accordance with an exemplary embodiment of the present invention. FIG. 3 illustrates how the hand (150) is sandwiched between the first cushion (104) and the second cushion (104) while in the closed position. The portion of netting (102) wraps around the entire handwich mitt (100) to also prevent the hand (150) from sliding out of the sides of the cushions (104, 106). With the handwich mitt (100) in place, the user's hand (150) is immobilized thereby preventing them from removing vital IVs, tubes, needles or other life sustaining equipment. Also, the handwich mitt (100) prevents the user from damaging themselves or others if they are violent or aggressive.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A dual cushion restraint mitt comprising:
a first cushion positioned above a hand of a user;
a second cushion attached to said first cushion at a seam, where said second cushion rests beneath said hand;
a portion of netting fastened to said first cushion and attached at said seam, wherein said portion of netting wraps around said first cushion and said second cushion; and
a cuff attached to an end of said portion of netting opposite to said seam, wherein said cuff wraps around a wrist of said user to keep said portion of netting in place while said first cushion and said second cushion sandwich said hand to restrain and to prevent said user from removing life sustaining equipment.

2. The dual cushion restraint mitt according to claim 1, wherein said cuff includes a strap and an attachment means to secure said cuff around said wrist.

3. The dual cushion restraint mitt according to claim 2, wherein said attachment means includes at least one of a “D” ring, a hook and loop fastener and a snap.

4. The dual cushion restraint mitt according to claim 1, wherein said dual cushion restraint mitt further prevents said user from damaging themselves and others.

5. The dual cushion restraint mitt according to claim 1, wherein said first cushion includes a plurality of foam pieces arranged to prevent said hand from escaping from between said first cushion and said second cushion.

6. The dual cushion restraint mitt according to claim 1, wherein said first cushion and said second cushion are round shaped.

7. The dual cushion restraint mitt according to claim 1, wherein said first cushion and said second cushion are square shaped.

8. The dual cushion restraint mitt according to claim 1, wherein said cuff is lined with a soft material.
